(19) United States
(12) Patent Application Publication
(10) Pub. No.: US 2024/0156717 A1
(43) Pub. Date: May 16, 2024

(54) COMPOSITION FOR ENHANCING SKIN WHITENING EFFECT ACCORDING TO AUTOPHAGY ACTIVITY WITH NYPA FRUTICANS EXTRACT AS AN ACTIVE INGREDIENT

(71) Applicant: KYUNGPOOK NATIONAL UNIVERSITY INDUSTRY - ACADEMIC COOPERATION FOUNDATION, Daegu (KR)

(72) Inventor: Sang Han Lee, Daegu (KR)

(73) Assignee: KYUNGPOOK NATIONAL UNIVERSITY INDUSTRY - ACADEMIC COOPERATION FOUNDATION, Daegu (KR)

(21) Appl. No.: 18/503,688

(22) Filed: Nov. 7, 2023

(30) Foreign Application Priority Data
Nov. 10, 2022 (KR) 10-2022-0149570

(51) Int. Cl. A61K 8/9794; A61K 36/889; A61P 17/00; A61Q 19/02

(52) U.S. Cl. CPC A61K 8/9794; A61K 36/889; A61P 17/00; A61Q 19/02

(57) ABSTRACT

The present disclosure relates to a composition for skin whitening containing a Nypa fruticans extract as an active ingredient. The composition has an effect of reducing melanin content and inhibiting melanogenesis, and enhances a skin whitening effect by enhancing autophagy activity, and thus can be used in a cosmetic composition, a health functional food composition, and a pharmaceutical composition.

### CROSS-REFERENCE TO RELATED APPLICATIONS

This application is based on and claims priority from Korean Patent Application No. 10-2022-0149570, filed on Nov. 10, 2022, with the Korean Intellectual Property Office, the disclosure of which is incorporated herein in its entirety by reference.

### TECHNICAL FIELD

The present disclosure relates to a composition for enhancing a skin whitening effect according to autophagy activity with a Nypa fruticans extract as an active ingredient.

### BACKGROUND

The most important factor involved in skin whitening is skin melanin. Melanin is a black pigment found in animals, plants, microorganisms, and the like, and is a substance that increases survival and competitiveness in a specific environment although it is not essential for growth or development. Melanin in animals is related to skin diseases and malignant melanoma, and in addition to DOPA melanin produced from tyrosine by tyrosinase, DHN melanin, GDHB melanin, catechol melanin, etc. have been reported.

When melanin is overproduced, melanin continues to be released out of the skin, causing freckles, or darkening of the skin in local areas. Accordingly, various whitening methods are attracting attention to prevent excessive deposition of melanin on the skin and reduce the pigment content in the skin. Main methods include UV blocking, a method for quickly bleaching a melanin pigment, a method of blocking a step in which biosynthesized melanin is loaded in melanosomes to be transferred to keratinocytes, a method of inhibiting tyrosinase activity, which is a rate-determining step in melanin biosynthesis, a method of blocking signaling that induces tyrosinase activity and biosynthesis promotion from the cell membrane of melanocytes, a method of administering substances that are specifically toxic to melanocytes, and the like.

To date, representative substances for inhibiting tyrosinase, which is known as an important factor in melanin synthesis, include arbutin, kojic acid, hydroquinone, vitamin C, and the like, and particularly, arbutin is known to have an effect of inhibiting melanosome production due to 5,6-dihydroxyindole-2-carboxylic acid (DHICA) polymerase activity in addition to a function of inhibiting tyrosinase activity. However, problems on the stability and safety of these substances and controversy over side effects have continued.

Meanwhile, the present disclosure confirms a skin whitening effect of a Nypa fruticans extract and provides a cosmetic composition, a health functional food composition, and a pharmaceutical composition using the same.

### PRIOR ARTS

### Patent Documents

(Patent Document 1) KR 10-2257307 B1

### SUMMARY

The present disclosure has been made in an effort to provide a skin whitening method including administering a composition containing a Nypa fruticans extract in an effective dose to a subject or topically applying the composition to the skin of the subject.

An embodiment of the present disclosure provides a skin whitening method including topically applying a composition containing a Nypa fruticans extract in an effective dose to the skin of a subject.

In the present disclosure, the Nypa fruticans extract may preferably use ethanol as an extract solvent.

In the present disclosure, the Nypa fruticans extract may preferably reduce the melanin content.

In the present disclosure, the Nypa fruticans extract may preferably inhibit melanogenesis.

In the present disclosure, the Nypa fruticans extract may preferably reduce the expression of TYR, TYRP-1, TYRP-2 and MITF.

In the present disclosure, the Nypa fruticans extract may preferably increase the expression of p-ERK, p-JNK, and p-p38.

In the present disclosure, the Nypa fruticans extract may preferably enhance a whitening effect according to autophagy activation.

Another embodiment of the present disclosure provides a skin whitening method including administering a composition containing a Nypa fruticans extract in an effective dose to a subject.

Yet another embodiment of the present disclosure provides a method for treating skin diseases caused by excessive melanin production, including administering a composition containing a Nypa fruticans extract as an active ingredient to a subject in need of treatment for skin diseases caused by excessive melanin production.

In the present disclosure, the skin diseases caused by excessive melanin production may be preferably one or more selected from the group consisting of melasma, freckles, age spots, blemishes, epidermal melanocytic lesions, Cafe's au lait macules, nevus, Becker's nevus, nevus spilus, lentigines, lentigo, dermal melanocytic lesions, mongolian spots, Nevus of Ota, acquired bilateral nevus of Ota-like macules, nevus of Ito, blue nevus, melanocytic nevus, junctional nevus, compound nevus, intradermal nevus, halonevus, congenital melanocytic nevus, Spitz nevus, dysplastic nevus, melanoma, lentigo maligna melanoma, superficial spreading melanoma, acral lentiginous melanoma, nodular melanoma, pigment basal cell carcinoma, pigment dermatofibroma, pigment dermoid cyst, pigment keloid, melanin deposition caused by ultraviolet rays, melanin deposition caused by drugs, melanin deposition after inflammation, melanin deposition occurring in dermatitis, and pigment keratoacanthoma.

According to the embodiments of the present disclosure, it is possible to provide a skin whitening method using a Nypa fruticans extract having effects of reducing the melanin content and inhibiting melanin formation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates results of a cytotoxicity evaluation experiment of a Nypa fruticans extract in an embodiment of the present disclosure.

FIG. 2 is a diagram confirming a melanin content reduction effect of a Nypa fruticans extract in an embodiment of the present disclosure.

FIG. 3 is a diagram confirming melanogenesis-related mRNA expression of a Nypa fruticans extract in an embodiment of the present disclosure.

FIG. 4 is a diagram confirming melanogenesis-related protein expression of a Nypa fruticans extract in an embodiment of the present disclosure.

FIG. 5 is a diagram confirming that a melanogenesis inhibitory effect of a Nypa fruticans extract is associated with MAPK signaling, in an embodiment of the present disclosure.

FIG. 6 is a diagram confirming that a melanogenesis inhibitory effect increases with combined treatment of a Nypa fruticans extract and an autophagy inducer in an embodiment of the present disclosure.

FIG. 7 is a diagram confirming that the inhibitory activity of melanogenesis-related protein increases with combined treatment of a Nypa fruticans extract and an autophagy inducer in an embodiment of the present disclosure.

FIG. 8 is a diagram confirming that a melanin content reduction effect increases with combined treatment of a Nypa fruticans extract and an autophagy inducer in an embodiment of the present disclosure.

FIG. 9 is a diagram illustrating an autophagy-related whitening activation mechanism of a Nypa fruticans extract in an embodiment of the present disclosure.

FIG. 10 is a diagram illustrating RSM and ANN analysis results for deriving optimal extraction conditions related to the tyrosinase inhibitory activity of a Nypa fruticans extract in an embodiment of the present disclosure.

FIG. 11 is a diagram illustrating RSM and ANN analysis results in an embodiment of the present disclosure, in which (a) of FIG. 11 is a diagram illustrating linear, quadratic and interaction terms of target parameters with coefficients for model prediction, and (b) of FIG. 11 is a diagram illustrating comparative error analysis of RSM and ANN models.

FIG. 12 is a diagram illustrating RSM and ANN analysis results in an embodiment of the present disclosure, in which (a) of FIG. 12 is a diagram illustrating a 3D response surface plot of a Nypa fruticans extract according to extraction conditions, (b) of FIG. 12 is a diagram illustrating an ANN model, and (c) of FIG. 12 is a diagram illustrating a network training curve for tyrosinase inhibitory activity (TIA) using MATLAB software.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

The present disclosure provides a skin whitening method including topically applying a cosmetic composition containing a Nypa fruticans extract in an effective dose to the skin of a subject.

Nypa fruticans is a plant belonging to the palm family and is the bamboo shoot of a palm tree called Nipa palm, which is distributed in places such as India. Pinnate leaves about 3 to 9 mm long from the rhizome that extends underground come out on the ground and grow straight. The distinction between roots and stems of Nypa fruticans is not clear, and the present disclosure used the roots and stems rather than flower buds of Nypa fruticans.

Meanwhile, melanin is observed in the outer skin, feathers, skin, hair, eyes, etc. of animals, and when the melanin is overproduced, the melanin is deposited on the skin to form spots and freckles, promote skin aging, and also cause skin cancer.

In the present disclosure, the Nypa fruticans extract may be obtained and used by extraction and separation from nature using extraction and separation methods known in the art. The “extract” as defined in the present disclosure is extracted from Nypa fruticans using an appropriate solvent, and includes, for example, a crude extract, a polar solvent-soluble extract, or a non-polar solvent-soluble extract. As a suitable solvent for extracting the extract from the Nypa fruticans, any food or pharmaceutically acceptable organic solvent may be used, and water or an organic solvent may be used, but is not limited thereto. For example, various solvents such as purified water, alcohols having carbon atoms 1 to 4 including methanol, ethanol, propanol, isopropanol, butanol, etc., acetone, ether, benzene, chloroform, ethyl acetate, methylene chloride, hexane, cyclohexane, and the like may be used alone or in combination. As an extraction method, any one of methods such as hot water extraction, chilling extraction, reflux cooling extraction, solvent extraction, steam distillation, ultrasonic extraction, elution, and compression may be selected and used. In addition, a desired extract may also be additionally subjected to a conventional fractionation process or may be purified using a conventional purification method.

There is no limitation on the preparation method of the extract of the present disclosure, and any known method may be used. For example, the extract included in the composition of the present disclosure may be prepared in a powder state by additional processes such as distilling under reduced pressure and freeze-drying or spray-drying of a primary extract extracted by the hot water extraction or solvent extraction method. In addition, fractions may also be obtained by further purifying the primary extract using various chromatography, such as silica gel column chromatography, thin layer chromatography, and high performance liquid chromatography. Accordingly, in the present disclosure, the extract is a concept that includes all extracts, and fractioned and purified products that are obtained in each step of extraction, fractionation, or purification, and dilutions, concentrates, or dried products thereof.

In one embodiment of the present disclosure, the skin whitening method includes (a) directly applying the cosmetic composition to the skin of a subject, (b) contacting or attaching a patch, mask pack, or mask sheet to which the cosmetic composition is applied or deposited to the skin of a subject, or sequentially performing (a) and (b) above. In step (a), lotions, creams, essences, etc. may be used as cosmetic compositions.

The “cosmetic composition” of the present disclosure may be prepared by containing a cosmetically effective amount of the extract extracted from the Nypa fruticans of the present disclosure described above and a cosmetically acceptable carrier.

In the present specification, the “cosmetically effective amount” means an amount sufficient to achieve a skin whitening effect of the present disclosure described above.

The appearance of the cosmetic composition contains a cosmetically or dermatologically acceptable medium or base. The appearance of the cosmetic composition may be provided in all formulations suitable for local application, for example, solutions, gels, solids, pasty anhydrous products, emulsions obtained by dispersing an oil phase in a water phase, suspensions, microemulsions, microcapsules, microgranules or ionic (liposomes) and non-ionic vesicular dispersions, or creams, toners, lotions, powders, ointments, sprays or concealer sticks. These compositions may be prepared according to conventional methods in the art. The composition according to the present disclosure may also be used in the form of a foam or in the form of an aerosol composition further containing a compressed propellant.

The cosmetic composition according to an embodiment of the present disclosure is not particularly limited in its formulation, and may be formulated in cosmetics, such as softening lotion, astringent lotion, nourishing lotion, nourishing cream, massage cream, essence, eye cream, eye essence, cleansing lotion, cleansing foam, cleansing water, pack, powder, body lotion, body cream, body oil, and body essence.

When the formulation of the cosmetic composition of the present disclosure is the paste, cream, or gel, as carrier ingredients, animal oils, vegetable oils, wax, paraffin, starch, tragacanth, cellulose derivatives, polyethylene glycol, silicone, bentonite, silica, talc, zinc oxide or the like may be used.

When the formulation of the cosmetic composition of the present disclosure is the powder or spray, as the carrier ingredients, lactose, talc, silica, aluminum hydroxide, calcium silicate or polyamide powder may be used, and particularly, in the case of the spray, a propellant such as chlorofluorohydrocarbon, propane/butane or dimethyl ether may be additionally included.

When the formulation of the cosmetic composition of the present disclosure is the solution or emulsion, as the carrier ingredients, a solvent, a solubilizing agent or an emulsifying agent may be used. For example, the carrier ingredients include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan.

When the formulation of the cosmetic composition of the present disclosure is the suspension, as the carrier ingredients, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the cosmetic composition of the present disclosure is the surfactant-containing cleansing, as the carrier ingredients, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

The cosmetic composition of the present disclosure may be applied with cosmetics, such as tonor, lotion, cream, essence, pack, foundation, color cosmetics, sunscreen, two-way cake, face powder, compact, makeup base, skin cover, eye shadow, lipstick, lip gloss, lip fix, and eyebrow pencil, and beauty wash, and cleaning agents, such as shampoo and soap.

The cosmetic composition according to an embodiment of the present disclosure may further include functional additives and ingredients included in general cosmetic compositions in addition to the extract extracted from the Nypa fruticans. The functional additives may include ingredients selected from the group consisting of water-soluble vitamins, oil-soluble vitamins, polymer peptides, polymer polysaccharides, sphingolipids, and seaweed extract.

In addition to the functional additives, the cosmetic composition of the present disclosure may also contain ingredients included in general cosmetic compositions, if necessary. Other mixed ingredients to be included may include oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulation accelerators, cooling agents, adiaphoretics, and purified water.

The present disclosure provides a skin whitening method including administering a food composition containing a Nypa fruticans extract in an effective dose to a subject.

The food composition of the present disclosure may be formulated into various forms such as tablets, pills, granules, capsules, liquid preparations, and beverages to be added to foods. The kind of food is not particularly limited. Examples of the food which may be added with the active substances include drinks, meat, sausages, bread, biscuits, rice cakes, chocolate, candies, snacks, confectionery, pizza, ramen, other noodles, gums, dairy products including ice cream, various soups, beverages, alcohol beverages and vitamin complexes, dairy products and processed dairy products, and the like, and include all health foods and health functional foods in an accepted meaning.

Meanwhile, in addition to containing the extract as the active ingredient, the food composition of the present disclosure may contain various flavoring agents, natural carbohydrates, or the like as additional ingredients like conventional food compositions.

Examples of the above-mentioned natural carbohydrates may include general sugars, such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, erythritol, and the like. The above-mentioned flavoring agents may be advantageously used with natural flavoring agents (tauumatin), stevia extract (e.g., rebaudioside A, glycyrrhizin, etc.), and synthetic flavoring agents (saccharin, aspartame, etc.). The food composition of the present disclosure may be formulated in the same manner as the pharmaceutical composition below to be used as a functional food or added to various foods. The food that may add the composition of the present disclosure may include, for example, beverages, meat, chocolate, foods, confectionery, pizza, ramen, other noodles, gums, candies, ice creams, alcoholic beverages, vitamin complexes, health food supplements, and the like.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present disclosure may contain natural fruit juice, and pulps for preparing fruit juice beverages and vegetable beverages.

The food composition of the present disclosure may be produced and processed in the form of tablets, capsules, powders, granules, liquids, and pills. The “health functional food” used herein is the same term as food for special health use (FoSHU), and refers to food with a high medical/care effect, which is processed so that a bioregulatory function is effectively shown in addition to nutrition supply. Here, the “function” refers to adjusting nutrients or to obtaining effects useful for health applications such as physiological action with respect to the structures and functions of the human body. The health functional food of the present disclosure is able to be prepared by methods which are commonly used in the art, and may be prepared by adding raw materials and ingredients which are commonly added in the art in preparation. In addition, formulations of the health functional food may also be prepared without limitations as long as the formulation is recognized as food. The health functional food of the present disclosure may be prepared in various types of formulations, and unlike general drugs, the health functional food has an advantage that it is made from food as a raw material and thus there is no side effect that may occur when taking a long-term use of the drug, and has excellent portability, so that the health functional food of the present disclosure is able to be taken as supplements to enhance the skin whitening effect. For example, the health functional food in the form of tablets may be formed by granulating a mixture obtained by mixing the active ingredient of the present disclosure with an excipient, a binder, a disintegrant, and other additives by a conventional method, and then compression-molding the mixture by adding a slip modifier and the like, or directly compressing the mixture. In addition, the health functional food in the form of tablets may also contain a flavors enhancer or the like as needed. In the health functional food in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture mixed with the active ingredient of the present disclosure and an excipient, a binder, a disintegrant, and the like by existing known methods, and may also be coated with white sugar or other coating agents or surface-coated with materials such as starch and talc, if necessary. The health functional food in the form of granules may be prepared by granulizing a mixture mixed with the active ingredient of the present disclosure and an excipient, a binder, a disintegrant, and the like by existing known methods and may contain a flavoring agent, a flavors enhancer, and the like, if necessary.

The present disclosure provides a method for treating skin diseases caused by excessive melanin production, including administering a pharmaceutical composition containing a Nypa fruticans extract as an active ingredient to a subject in need of treatment for skin diseases caused by excessive melanin production.

In the present disclosure, the skin diseases caused by excessive melanin production may one or more selected from the group consisting of melasma, freckles, age spots, blemishes, epidermal melanocytic lesions, Cafe's au lait macules, nevus, Becker's nevus, nevus spilus, lentigines, lentigo, dermal melanocytic lesions, mongolian spots, Nevus of Ota, acquired bilateral nevus of Ota-like macules, nevus of Ito, blue nevus, melanocytic nevus, junctional nevus, compound nevus, intradermal nevus, halonevus, congenital melanocytic nevus, Spitz nevus, dysplastic nevus, melanoma, lentigo maligna melanoma, superficial spreading melanoma, acral lentiginous melanoma, nodular melanoma, pigment basal cell carcinoma, pigment dermatofibroma, pigment dermoid cyst, pigment keloid, pigment (melanin) deposition caused by ultraviolet rays, pigment (melanin) deposition caused by drugs, pigment (melanin) deposition after inflammation, pigment (melanin) deposition occurring in dermatitis, and pigment keratoacanthoma.

In addition, the pharmaceutical composition of the present disclosure may further include an adjuvant in addition to the active ingredient. Any adjuvant known in the art may be used without limitation.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, and sterile injectable solutions according to a conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one excipient, for example, starch, calcium carbonate, sucrose, lactose, gelatin, and the like with the active ingredient. Further, lubricants such as magnesium stearate and talc may be used in addition to simple excipients. Liquid formulations for oral administration may correspond to suspensions, oral liquids, emulsions, syrups, and the like, and may include various excipients, for example, a wetting agent, a sweetener, an aromatic agent, a preserving agent, and the like, in addition to water and liquid paraffin which are commonly used diluents. Formulations for parenteral administration include sterilized aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, and the like may be used. As a base of the suppository, witepsol, Tween 61, cacao butter, laurinum, glycerogelatin, and the like may be used.

The pharmaceutical composition of the present disclosure may be administered to a subject through various routes. All methods of administration may be expected, and the pharmaceutical composition may be administered, for example, by oral, intravenous, intramuscular, subcutaneous, and intraperitoneal injection.

The pharmaceutical composition of the present disclosure may be formulated into various oral or parenteral dosage forms.

Formulations for oral administration include, for example, tablets, pills, hard and soft capsules, solutions, suspensions, emulsifiers, syrups, granules, etc., and these formulations may further include diluents (e.g., lactose, dextrose, sucrose, mannitol, sorbitol, cellulose and/or glycine), lubricants (e.g., silica, talc, stearic acid and its magnesium or calcium salts and/or polyethylene glycol), in addition to the active ingredient. In addition, the tablets may contain a binder such as magnesium aluminum silicate, starch paste, gelatin, tragacanth, methylcellulose, sodium carboxymethylcellulose and/or polyvinylpyrrolidine, and in some cases, may contain disintegrants or effervescent mixtures such as starch, agar, alginic acid or its sodium salt and/or an absorbent, a coloring agent, a flavoring agent and a sweetening agent. The formulations may be prepared by conventional mixing, granulating or coating methods.

In addition, a typical formulation for parenteral administration is an injection formulation, and as a solvent for the injection formulation, water, a Ringer's solution, isotonic physiological saline or a suspension may be used. Sterile fixed oils of the injection formulation may be used as a solvent or suspension medium, and any non-irritating fixed oil including mono- and di-glycerides may be used for this purpose.

In addition, the injection formulation may use fatty acids such as oleic acid.

All results were expressed as mean±SD of at least three independent experiments and determined by one-way analysis of variance with a Dunnett's multiple comparison test using SigmaPlot Software V. 12.5 (Systat Software Inc.). Statistical significance was set at *P<0.05 and **P<0.01. In addition, statistical analysis of RSM results was performed using Design Expert software V. 11 (Stat-Ease Inc.).

Hereinafter, the present disclosure will be described in more detail through Examples and Experimental Examples. However, the following Examples and Experimental Examples are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

### <Example 1> Preparation of Nypa fruticans Extract

30 g of dried Nypa fruticans powder was mixed with 300 mL of 100% (v/v) ethanol, extracted in a shaking incubator at 50° C. for 16 hours, and then concentrated at 40° C. and 50 rpm using a vacuum rotary evaporator (Tokyo Rikakikai Co. Ltd., Japan) and freeze-dried for 3 days using a freeze dryer (Ilshin Biobase, Korea) to prepare a Nypa fruticans extract.

### <Example 2> Evaluation of Cytotoxicity

Mouse melanocytes (Melan-a) were obtained from Dorothy C Bennett (St George's, University of London, London). The cells were cultured in RPMI 1640 medium (Cellgro, USA) containing 10% fetal bovine serum (Hyclone, Utah, UT, USA), 100 μg/mL of streptomycin-penicillin, and 200 nM potent tumor promoter (TPA), and were maintained at 5% CO₂, and 37° C. The cells were subcultured when 80% confluency cells were reached.

Cytotoxicity of the Nypa fruticans extract was analyzed using an MTT method based on formazan formation. The cells were inoculated in a 96-well plate at 1×10⁵ cells/well for 24 hours. After 24 hours, the cells were treated with the Nypa fruticans extract at different concentrations and cultured. After reacting at 37° C. for 30 minutes, an MTT solution (0.5 mg/mL) was mixed with each cell. After the medium was removed, the plate was washed twice with PBS at pH 7.4, and insoluble intracellular formazan was solubilized with DMSO. The absorbance of the produced formazan was analyzed at 595 nm using a spectrophotometer.

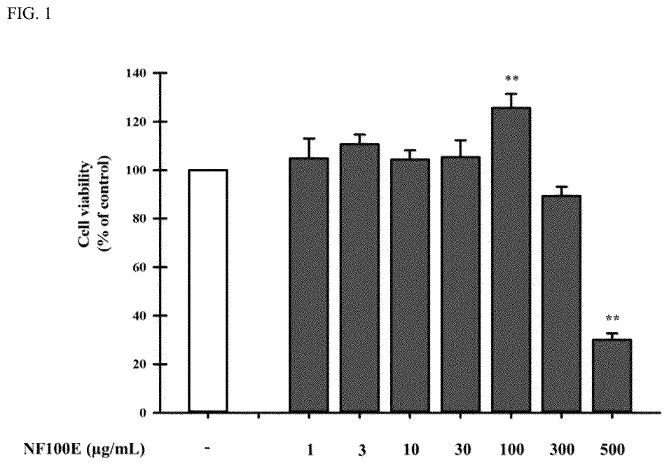

As shown in FIG. 1, it was confirmed that the Nypa fruticans extract of the present disclosure had no cytotoxicity up to 300 μg/mL, and hereinafter, the Nypa fruticans extract at a concentration of 100 μg/mL was used.

### <Example 3> Skin Whitening Effect of Nypa fruticans Extract

Mouse melanocytes (Melan-a) were inoculated into a 24-well plate at 1×10⁵ cells/mL and cultured overnight. Thereafter, the medium was replaced with a new medium containing 100 μM IBMX (melanogenesis promoter) and 10 μg/mL and 100 μg/mL of the Nypa fruticans extract of the present disclosure, and reacted for 72 hours, and the existing medium was washed. Thereafter, the cells were washed twice with PBS and dissolved in 1 N NaOH.

3-1. Melanin Content Analysis

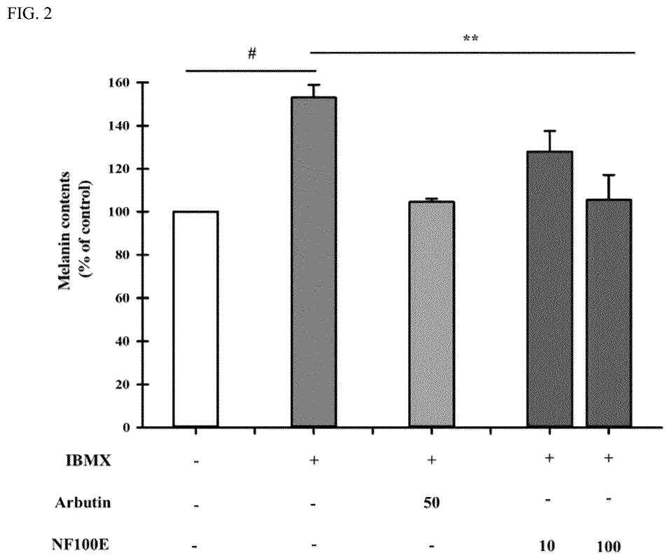

The melanin content was analyzed at 405 nm with a spectrophotometer, and arbutin was used as a positive control group. As shown in FIG. 2, IBMX treatment increased the melanin content by about 1.5 times compared to an IBMX-untreated control group. However, when treated with 100 μg/mL of the Nypa fruticans extract of the present disclosure, a melanin content reduction effect similar to that of the arbutin-treated group, which was the positive control group, was confirmed.

3-2. Melanogenesis Inhibitory Effect (RT-PCR)

In melanogenesis, tyrosinase (TYR), tyrosinase-related protein-1 (TYRP-1) and tyrosinase-related protein-2 (TYRP-2) were key factors. An increase in the intracellular concentration of cyclic adenosine monophosphate (cAMP) as a secondary messenger was stimulated by α-MSH with adenylyl cyclase after binding to MC1R, and the activation of cAMP triggered protein kinase A (PKA) along with phosphorylation of a cAMP response element (CREB) protein linked to an M-box motif in the promoter region to upregulate a transcription factor MITF and a melanogenic enzyme. In the experiment, phosphorylated CREB was evaluated as a transcription factor and melanogenesis-related genes such as tyrosinase, TYRP-1, TYRP-2, MITF, and MC1R in mouse melanocytes (Melan-a) to confirm a melanogenesis inhibitory effect.

Total RNA (2 μg) was extracted from the mouse melanocytes (Melan-a) using TRIzol (Invitrogen Co., Carlsbad, CA, USA). Total RNA was used as a template to synthesize cDNA using RT-&GO Mastermix (MP Biomedicals, Seoul, Korea). RT-PCR was performed using PCR Thermal Cycler Dice TP600 (TAKARA Bio Inc., Otsu, Japan). The resulted products were separated by gel electrophoresis on a 1% agarose gel at 100 V for 30 minutes and stained using ethidium bromide (Bid-Rad Laboratories, Hercules, CA, USA). Quantification of bands was calculated using Image Lab™ software version 5.2.1 (Bio-Rad Laboratories, CA, USA).

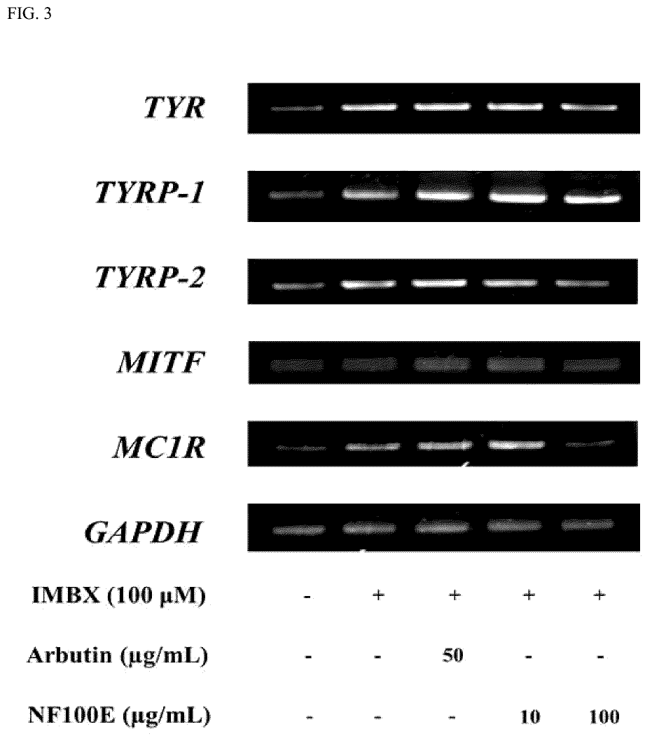

As shown in FIG. 3, melanogenesis-related mRNA expression was increased with IBMX treatment, but when treated with the Nypa fruticans extract of the present disclosure, the expression levels of TYR, TYRP-2, and MITF were significantly inhibited.

3-3. Melanogenesis Inhibitory Effect (Western Blot)

Mouse melanocytes (Melan-a) were lysed with a RIPA buffer (ELPIS Biotech. Inc., Korea) containing protease inhibitor cocktail I (P8340; Sigma-Aldrich Korea) and phosphatase inhibitor cocktail II (P5726; Sigma-Aldrich Korea) and the extracted proteins were quantified using a Bradford protein method. Each protein was separated by 10% SDS-PAGE, and then transferred to a nitrocellulose (NC) membrane (0.45 μm; 1620115; Bio-Rad, Munich, Germany) and blocked with 5% skim milk or BSA in TBST for 1 hour. The membrane was cultured overnight with primary antibodies at 4° C. The membrane was washed four times at 15-minute intervals, and then reacted with anti-mouse or rabbit IgG-HRP as a secondary antibody. The proteins were detected using the ECL Solution System (ChemiDoc™ XRS+; Bio-Rad), and protein bands were quantified using Image Lab™ software (Bio-Rad Laboratories, CA, USA).

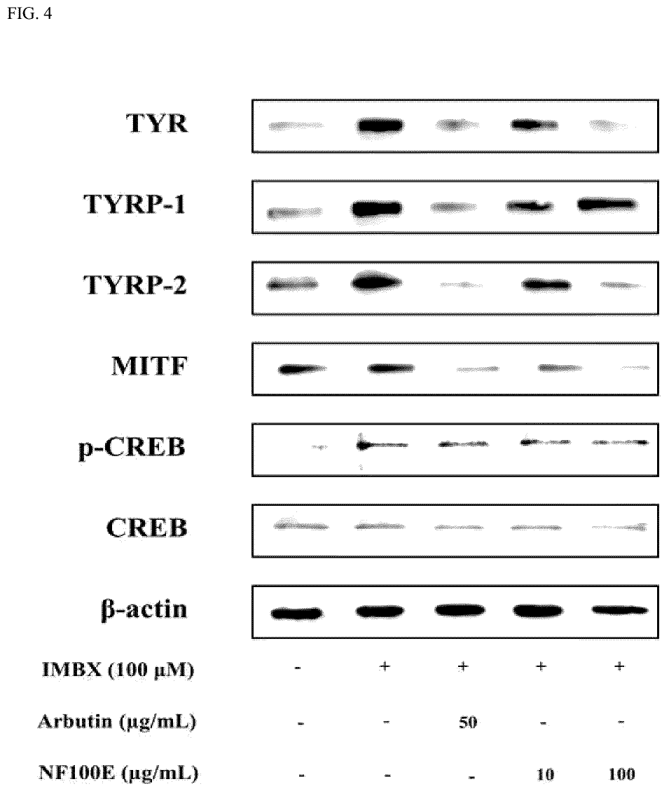

As shown in FIG. 4, melanogenesis-related protein levels were increased with IBMX treatment, but when treated with the Nypa fruticans extract of the present disclosure, the expression levels of TYR, TYRP-2, and MITF were significantly inhibited.

Meanwhile, cAMP-dependent protein kinase (PKA), mitogen-activated protein kinase (ERK, JNK, p38), phosphatidylinositol 3-kinase (PI3K)/AKT, and the like were known to regulate melanin pigment formation and melanogenesis-related genes. MAP kinase-mediated signaling caused ubiquitination and degradation of MITF, thereby reducing melanogenesis. Therefore, in order to confirm the effect of the Nypa fruticans extract of the present disclosure on a melanogenesis inhibition mechanism, Western blot analysis was performed on phosphorylation at the molecular level of MAPK signaling in mouse melanocytes (Melan-a).

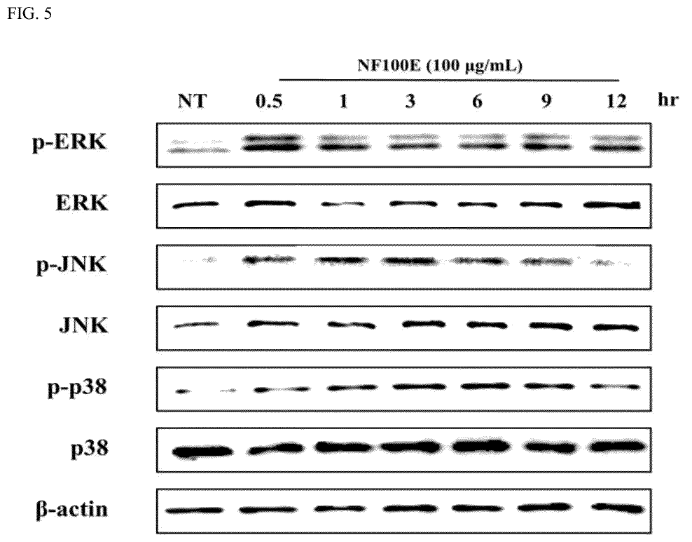

As shown in FIG. 5, the Nypa fruticans extract of the present disclosure activated p-ERK, p-JNK, and p-p38. In other words, it was confirmed that the melanogenesis inhibitory effect by the Nypa fruticans extract of the present disclosure was related to MAPK signaling.

### <Example 4> Enhancement of Whitening Effect of Nypa fruticans Extract According to Autophagy Activity

Autophagy activity is known to play an important role in regulating melanogenesis. The expression of autophagy-related proteins such as LC3B, Beclin-1, and ATG12 has been reported to inhibit melanogenesis in melanoma patients, and decreased MITF protein has been reported to suggest inhibition of melanogenesis. Accordingly, Western blot analysis was performed to confirm whether the Nypa fruticans extract of the present disclosure inhibited melanogenesis by inhibiting the expression of autophagy-related proteins.

Mouse melanocytes (Melan-a) were inoculated into a 24-well plate at 1×10⁵ cells/mL and cultured overnight. Thereafter, the medium was replaced with a new medium containing 100 μM IBMX (melanogenesis promoter) and 10 μg/mL and 100 μg/mL of the Nypa fruticans extract of the present disclosure, and reacted for 72 hours. Thereafter, the cells were treated with rapamycin (autophagy inducer) or bafilomycin (autophagy inhibitor), and the melanin content was evaluated.

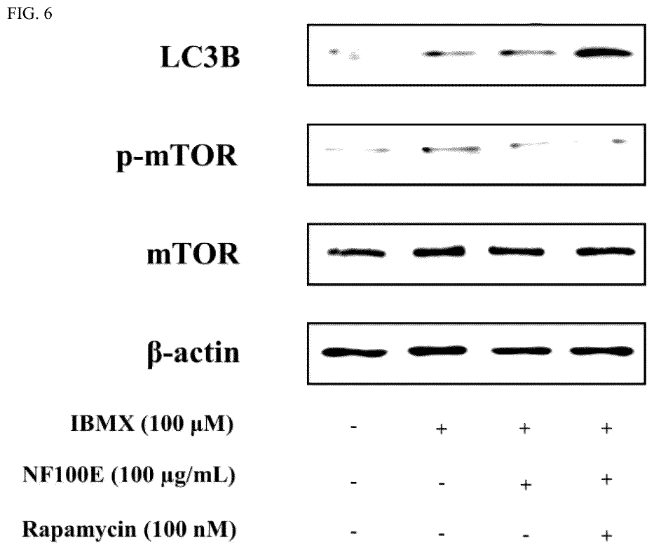

As shown in FIG. 6, it was confirmed that when the rapamycin (autophagy inducer) and the Nypa fruticans extract were treated simultaneously, the expression of LC3B was increased, and the expression of p-mTOR was decreased. That is, it was confirmed that the induction of autophagy activity and treatment with the Nypa fruticans extract of the present disclosure effectively increased LC3B protein expression, thereby exhibiting a melanogenesis inhibitory effect.

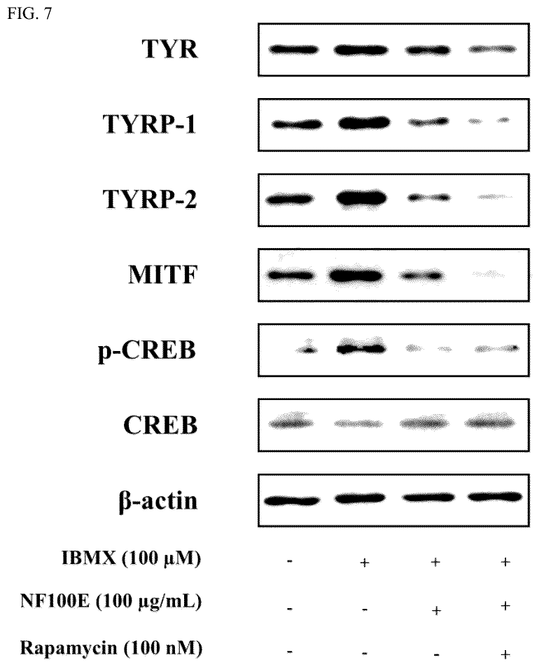

As shown in FIG. 7, the Nypa fruticans extract of the present disclosure inhibited melanogenesis-related proteins TYR, TYRP-1, TYRP-2, MITF, and p-CREB. When the rapamycin was used together as an autophagy inducer, the protein inhibitory activity as described above increased.

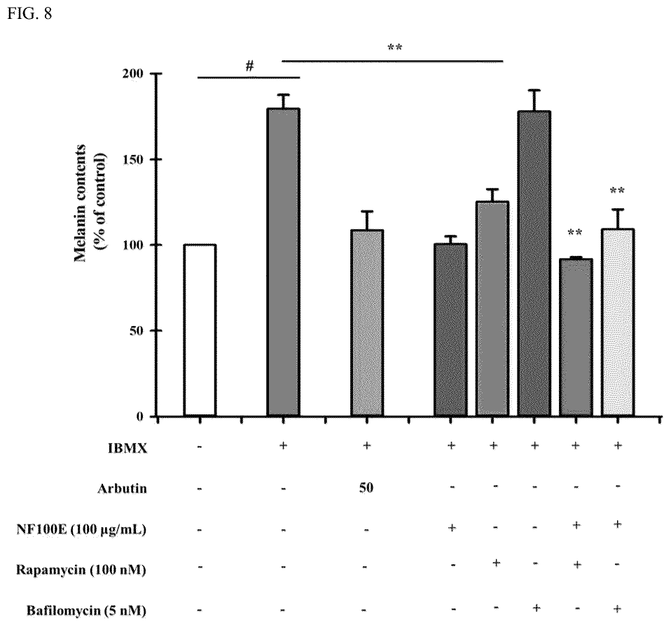

FIG. 8 is a diagram analyzing the melanin content according to treatment with rapamycin (autophagy inducer) and bafilomycin (autophagy inhibitor). As shown in FIG. 8, it was confirmed that the Nypa fruticans extract of the present disclosure reduced the melanin content, and the melanin reduction effect increased with simultaneous treatment with rapamycin. On the other hand, bafilomycin treatment did not affect the melanin content. In other words, it was confirmed that the Nypa fruticans extract of the present disclosure induced the formation of autophagosomes according to an effect of enhancing autophagy activity with rapamycin as a key autophagic molecule, and exhibited a more effective melanin content reduction effect to further improve a whitening effect.

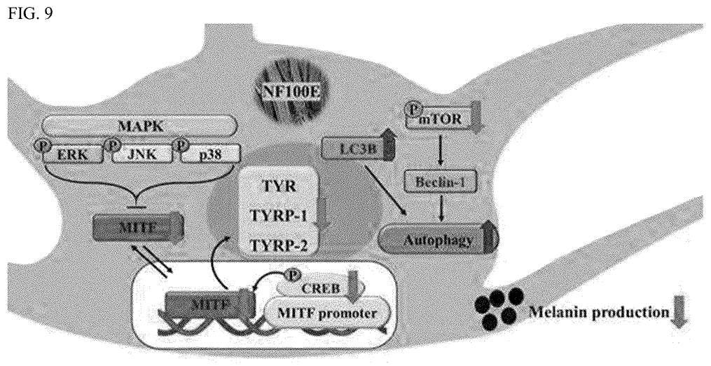

FIG. 9 is a diagram illustrating a mechanism of the Nypa fruticans extract of the present disclosure.

### <Example 5> Derivation of Optimal Extraction Conditions of Nypa fruticans Extract

RSM and ANN analysis was performed to derive optimal extraction conditions for the Nypa fruticans extract, which maximized tyrosinase inhibitory activity (TIA).

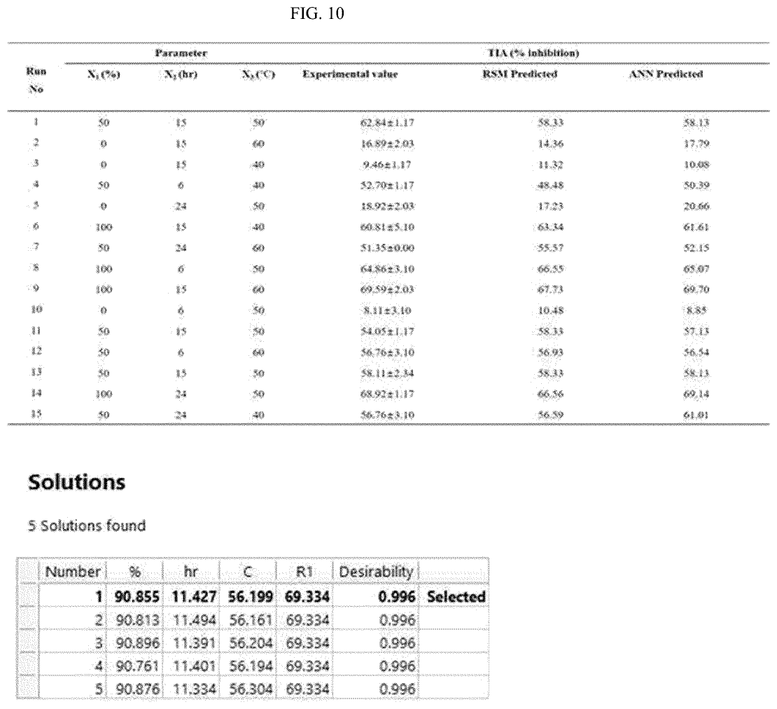

Main parameters were illustrated in FIG. 10. For RSM analysis, an ethanol concentration (X₁, 0% to 100%), an extraction time (X₂, 6 hours to 24 hours), and an extraction temperature (X₃, 40° C. to 60° C.) were set as independent parameters. The analysis was performed by setting the three extraction factors to 15 factors as shown in FIG. 10 according to Design Expert Software (Version 11; Stat-Ease Inc., Minneapolis, MN, USA). In addition, a dependent parameter affected by the independent parameter was tyrosinase inhibitory activity (TIA) (%), which was measured three times and the average value thereof was used. An interaction effect of each independent parameter on a response value was shown through a 3D surface diagram. For ANN analysis, an ethanol concentration (X₁, 0% to 100%), an extraction time (X₂, 6 hours to 24 hours), and an extraction temperature (X₃, 40° C. to 60° C.) were used as input parameters, and an output response was analyzed for tyrosinase inhibitory activity (TIA) (%). Multilayer Perceptron (MLP) and back-propagation feed-forward algorithm models were used for nonlinear analysis by MATLAB's Neural Network Toolbox™. Analysis of variance (ANOVA) was performed to analyze statistical significance and each term of a fitting model.

A second-order polynomial model was calculated by Equation 1 below. Y represents a dependent parameter for the independent reaction (X₁-X₃), and b₀, bᵢ, bᵢᵢ, and bᵢⱼ are constant coefficients of intercept, linear, quadratic, and interaction terms.

Y=b₀+Σᵢ₌₁³bᵢXᵢ+Σᵢ₌₁³bᵢᵢXᵢⱼ+Σ_{i<1}³bᵢⱼXᵢXⱼ [Equation 1]

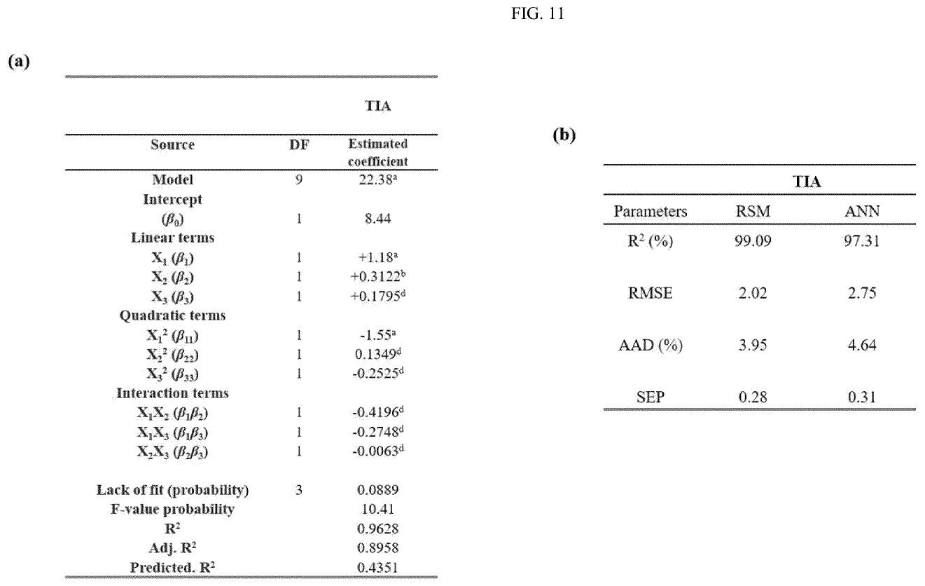

FIG. 10 illustrates a tyrosinase inhibitory activity (TIA) (%) result according to each condition. The experimental values of each extraction condition were similar to RSM and ANN predicted values. (a) of FIG. 11 is a diagram illustrating linear, quadratic and interaction terms of target parameters with coefficients for model prediction, and (b) of FIG. 11 is a diagram illustrating the comparative error analysis of RSM and ANN models.

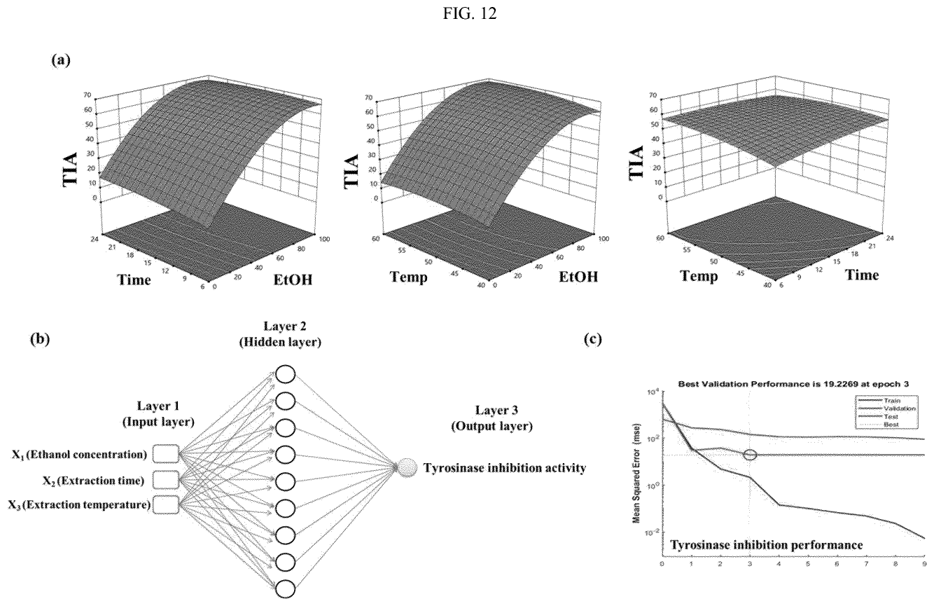

(a) of FIG. 12 is a diagram illustrating a 3D response surface plot of a Nypa fruticans extract according to extraction conditions, (b) of FIG. 12 is a diagram illustrating a ANN model, and (c) of FIG. 12 is a diagram illustrating a network training curve for tyrosinase inhibitory activity (TIA) using MATLAB software. The optimal extraction conditions for the Nypa fruticans extract, which showed the maximum tyrosinase inhibitory activity (TIA) derived through the above analysis, were derived as ethanol concentration (X₁) 90.855%, extraction time (X₂) 11.427 h, and extraction temperature (X₃) 56.199° C. (see FIG. 10), and the tyrosinase inhibitory activity (TIA) predicted under these extraction conditions was 67.73% (RSM) and 69.70% (ANN).

### <Example 6> Tyrosinase Inhibitory Activity of Nypa fruticans Extract According to Optimal Extraction Conditions

A Nypa fruticans extract was prepared according to ethanol concentration (X₁) 90.855%, extraction time (X₂) 11.427 h, and extraction temperature (X₃) 56.199° C. Tyrosinase inhibitory activity (TIA) was analyzed by reacting each Nypa fruticans extract with 200 μL of phosphate buffer 0.1 M (pH 6.5), 1 mM L-tyrosine, and 200 units/mL of mushroom tyrosinase in a 96 well plate and measuring the absorbance at 490 nm at 2-minute intervals while reacting at 37° C. for 40 minutes.

As a result of the experiment, the tyrosinase inhibitory activity of the Nypa fruticans extract under optimal extraction conditions was derived as 69.59%, which was similar to the value predicted in Example 5.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for whitening skin comprising topically applying a composition containing a Nypa fruticans extract in an effective dose to a skin of a subject.

2. The skin whitening method of claim 1, wherein the Nypa fruticans extract is extracted by ethanol used as an extraction solvent.

3. The skin whitening method of claim 1, wherein the Nypa fruticans extract reduces a melanin content.

4. The skin whitening method of claim 1, wherein the Nypa fruticans extract inhibits melanogenesis.

5. The skin whitening method of claim 1, wherein the Nypa fruticans extract reduces expressions of TYR, TYRP-1, TYRP-2 and MITF.

6. The skin whitening method of claim 1, wherein the Nypa fruticans extract increases expressions of p-ERK, p-JNK, and p-p38.

7. The skin whitening method of claim 1, wherein the Nypa fruticans extract enhances a whitening effect by activating autophagy.

8. A method for whitening skin comprising administering a composition containing a Nypa fruticans extract in an effective dose to a subject.

9. A method for treating skin diseases caused by excessive melanin production, comprising administering a composition containing a Nypa fruticans extract as an active ingredient to a subject in need of treatment for the skin diseases caused by excessive melanin production,
wherein the skin diseases caused by excessive melanin production are one or more selected from the group consisting of melasma, freckles, age spots, blemishes, epidermal melanocytic lesions, Cafe's au lait macules, nevus, Becker's nevus, nevus spilus, lentigines, lentigo, dermal melanocytic lesions, mongolian spots, Nevus of Ota, acquired bilateral nevus of Ota-like macules, nevus of Ito, blue nevus, melanocytic nevus, junctional nevus, compound nevus, intradermal nevus, halonevus, congenital melanocytic nevus, Spitz nevus, dysplastic nevus, melanoma, lentigo maligna melanoma, superficial spreading melanoma, acral lentiginous melanoma, nodular melanoma, pigment basal cell carcinoma, pigment dermatofibroma, pigment dermoid cyst, pigment keloid, melanin deposition caused by ultraviolet rays, melanin deposition caused by drugs, melanin deposition after inflammation, melanin deposition occurring in dermatitis, and pigment keratoacanthoma.
